(19) United States
(12) Patent Application Publication
(10) Pub. No.: US 2025/0290895 A1
(43) Pub. Date: Sep. 18, 2025

(54) COMPOSITIONS AND METHODS THAT REDUCE PRUSSIAN BLUE FORMATION DURING NANOPORE SEQUENCING

(71) Applicant: Roche Sequencing Solutions, Inc., Pleasanton, CA (US)

(72) Inventors: Masa Absec, Campbell, CA (US); Aykezar Adil, San Jose, CA (US); Cynthia Cech, Newcastle, WA (US); Santi Chrisanti, Santa Clara, CA (US); Peter Crisalli, Santa Clara, CA (US); Zelia Hunts, Berkeley, CA (US); Cara Machacek, Seattle, CA (US); Karla Munoz, Oakley, CA (US)

(21) Appl. No.: 18/862,479

(22) PCT Filed: May 2, 2023
(86) PCT No.: PCT/EP2023/061457

§ 371 Date: Nov. 1, 2024

Related U.S. Application Data
(60) Provisional application No. 63/337,413, filed on May 2, 2022.

(51) Int. Cl. G01N 27/447; C12Q 1/6869; C22C 19/03

(52) U.S. Cl. CPC G01N 27/44791; C12Q 1/6869; C22C 19/03; G01N 27/4473; G01N 27/44747

(57) ABSTRACT

This application discloses electrochemical cells, nanopore devices, and associated buffer compositions useful for nanopore-based nucleic acid sequencing. Also disclosed are methods for using the electrochemical cells, devices, and compositions in nanopore-based nucleic acid sequencing methods, such as nanopore Sequencing-by-Expansion (Nano-SBX) and nanopore Sequencing-by-Synthesis (Nano-SBS) methods.

### FIELD

This application relates to electrochemical cells and associated buffer compositions that reduce deleterious formation of Prussian Blue during nanopore-based assays, and methods for using the composition in nanopore-based nucleic acid detection techniques, such as nanopore sequencing.

### BACKGROUND

Nanopore-based nucleic acid sequencing is a compelling approach that has been widely studied. In one approach, the sequence a single-stranded polynucleotide is detected by changes in ionic current flow as the polynucleotide translocates through a nanopore embedded in a lipid bilayer that separates two sides of a electrochemical cell. During the polynucleotide's translocation partial blockage of the nanopore aperture alters the ionic flow over time resulting in changes in current that can be measured by the cell.

Sequencing by Expansion (“SBX”) is a nanopore-based nucleic acid sequencing method that uses a biochemical process to transcribe the sequence of DNA onto a measurable polymer molecule referred to as an “Xpandomer.” See e.g., U.S. Pat. No. 7,939,259, entitled, “High Throughput Nucleic Acid Sequencing by Expansion;” and PCT publication WO2020236526A1, entitled “Translocation control elements, reporter codes, and further means for translocation control for use in nanopore sequencing.” In the SBX process, a target nucleic acid sequence is encoded along the backbone Xpandomer sequence with reporter constructs that are separated by ˜10 nm that are designed to provide high-signal-to-noise, well-differentiated response signals during nanopore translocation. The enhanced signal-to-noise provided by the different response signals provides significantly increased sequence read efficiency and accuracy of Xpandomers relative to native nucleic acid molecules.

Nanopore-based sequencing-by-synthesis (“SBS”) uses a polymerase (or other strand-extending enzyme) covalently linked to a nanopore to synthesize a DNA strand complementary to a target sequence template (i.e., a copy strand). The nanopore embedded in a membrane in an electrochemical cell is used to concurrently detect the identity of each nucleotide monomer as it is added it to that growing strand. See e.g., US Pat. Publ. Nos. 2013/0244340 A1, 2013/0264207 A1, 2014/0134616 A1, 2015/0368710 A1, and 2018/0057870 A1, and published International Application WO 2019/166457 A1. Each added nucleotide monomer is detected by monitoring signals due to changes in ion flow through the nanopore as a tag moiety attached to each added nucleotide monomer enters the nanopore and alter the ion flow. For optimal performance, the tag moiety should reside in the nanopore for a sufficient amount of time to provide for a detectable, identifiable, and reproducible signal associated with altering ion flow through the nanopore (relative to the baseline “open current” flow), such that the specific nucleotide associated with the tag can be distinguished unambiguously from the other tagged nucleotides in the SBS solution.

Nanopore-based sequencing, however, are burdened by having to resolve small current signal differences immersed in significant background noise in a micro-volume electrochemical cell. This measurement challenge is complicated by small changes in the materials and parameters affecting the electrochemical cell including but not limited to, the electrode material, the electrochemical cell material, electrochemical solution buffer salts, and redox active reagents, pH, voltage, temperature, and viscosity.

Accordingly, there remains a need for electrochemical cell designs and materials, buffer and redox reagent compositions, and methods for using them to reduce or prevent formation of deleterious side-products (e.g., Prussian Blue precipitant) that interfere with accurate electrochemical signal detection in nanopore-based nucleic acid sequencing devices, systems, and methods.

### SUMMARY

The present disclosure relates generally to electrochemical cells and associated buffer compositions used in the cells that reduce deleterious formation of Prussian Blue, or related precipitants, during nanopore-based assays, as well as methods for using the cells and compositions for nanopore-based nucleic acid detection techniques, such as nanopore sequencing. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides an electrochemical cell comprising: (a) a nanopore embedded in a membrane that separates the cell into cis and trans chambers operably connected by the nanopore, wherein the cis and trans chambers each contain an electrode, a solution comprising ferrocyanide ion, ferricyanide ion, and a buffer composition; and (b) inlet and an outlet ports operably connected to the cell, wherein the ports comprise a metal.

In at least one embodiment of the electrochemical cell of the present disclosure, the metal exhibits a current density of less than or equal to 10⁻³ mA/cm² when polarized to a potential of about 0.3 V versus a Ag/AgCl reference electrode in a buffer solution of 1 M NH₄Cl, 800 mM urea, 100 mM HEPES, pH 7.4 or in a buffer solution of 1 M NH₄Cl, 800 mM urea, 100 mM MES at pH 6.2-6.8. In at least one embodiment, the metal contains less than 10% iron, less than 8% iron, less than 6% iron, or less than 5% iron. In at least one embodiment, the metal is a nickel alloy. In at least one embodiment, the metal is a nickel alloy selected from C276, C22, 625, and MP35N.

In at least one embodiment of the electrochemical cell of the present disclosure, the cell encloses a solution volume of between about 0.1 and 1000 femtoliters.

In at least one embodiment of the electrochemical cell of the present disclosure, the ferrocyanide and ferricyanide ions are at a total concentration of between about 25 mM and 250 mM.

In at least one embodiment of the electrochemical cell of the present disclosure, the buffer composition comprises ammonium acetate at a concentration of from about 0 mM to about 1500 mM. In at least one embodiment, the buffer composition comprises ammonium chloride at a concentration of less than 2000 mM. In at least one embodiment, the buffer composition comprises ammonium acetate at a concentration of from about 0 mM to about 1500 mM and an ammonium chloride at a concentration of less than about 2000 mM.

In at least one embodiment of the electrochemical cell of the present disclosure, the application of an alternating current (AC) to the cell with a voltage range of 450 mV to 1200 mV resulting in no visible formation of Prussian Blue in the cell for at least 1 hour, at least 2 hours, at least 4 hours, at least 6 hours, at least 8 hours, or at least 10 hours.

In at least one embodiment the present disclosure provides a method for sequencing a target nucleic acid, wherein the method comprises:
(a) providing an electrochemical cell comprising (i) a nanopore embedded in a membrane that separates the cell into cis and trans chambers operably connected by the nanopore, wherein the cis and trans chambers each contain an electrode, a solution comprising ferrocyanide ion, ferricyanide ion, and a buffer composition; and (ii) inlet and outlet ports comprising a metal operably connected to the cell;
(b) adding a molecule derived from a target nucleic acid to the cis chamber;
(c) applying a voltage to the cell that causes at least a portion of the molecule translocate to through the nanopore; and
(d) detecting changes in voltage flow in the cell as the molecule translocates through the nanopore, wherein the changes in voltage flow are indicative of a sequence of the target nucleic acid.

In at least one embodiment of the method for sequencing of the present disclosure, the metal exhibits a current density of less than or equal to 10⁻³ mA/cm² when polarized to a potential of about 0.3 V versus a Ag/AgCl reference electrode in a buffer solution of 1 M NH₄Cl, 800 mM urea, 100 mM HEPES, pH 7.4 or in a buffer solution of 1 M NH₄Cl, 800 mM urea, 100 mM MES at pH 6.2-6.8. In at least one embodiment, the metal contains less than 10% iron, less than 8% iron, less than 6% iron, or less than 5% iron. In at least one embodiment, the metal is a nickel alloy. In at least one embodiment, the metal is a nickel alloy selected from C276, C22, 625, and MP35N.

In at least one embodiment of the method for sequencing of the present disclosure, the cell encloses a solution volume of between about 0.1 and 1000 femtoliters.

In at least one embodiment of the method for sequencing of the present disclosure, the ferrocyanide and ferricyanide ions are at a total concentration of between about 25 mM and 250 mM.

In at least one embodiment of the method for sequencing of the present disclosure, the buffer composition comprises ammonium acetate at a concentration of from about 0 mM to about 1500 mM. In at least one embodiment, the buffer composition comprises ammonium chloride at a concentration of less than 2000 mM. In at least one embodiment, the buffer composition comprises ammonium acetate at a concentration of from about 0 mM to about 1500 mM and an ammonium chloride at a concentration of less than 2000 mM. In at least one embodiment, the concentration of ammonium acetate is 0 mM and the concentration of ammonium chloride is about 600 mM.

In at least one embodiment of the method for sequencing of the present disclosure, the application of an alternating current (AC) to the cell with a voltage range of 450 mV to 1200 mV resulting in no visible formation of Prussian Blue in the cell for at least 1 hour, at least 2 hours, at least 4 hours, at least 6 hours, at least 8 hours, or at least 10 hours.

In at least one embodiment of the method for sequencing of the present disclosure, the applied voltage comprises a baseline voltage; optionally, wherein the baseline voltage of from about 55 mV to about 95 mV. In at least one embodiment, the applied voltage comprises a pulse voltage; optionally, wherein the pulse voltage of from about 320 mV to about 550 mV. In at least one embodiment, the pulse voltage has a duration from about 5 us to about 10 μs. In at least one embodiment, the time between pulse voltages is from about 0.5 ms to about 1.7 ms. In at least one embodiment, the applied voltages provides an alternating current; optionally, wherein the alternating current has a periodicity of about 0.4 s to about 6 s.

In at least one embodiment of the method for sequencing of the present disclosure, the molecule derived from the target nucleic acid comprises an Xpandomer. In at least one embodiment, the Xpandomer comprises a plurality of XNTP subunits coupled in a sequence corresponding to a contiguous nucleotide sequence of all or a portion of the target nucleic acid, wherein the individual XNTP subunits of the strand comprise a reporter construct, a nucleobase residue, and a selectively cleavable bond, and wherein cleavage of the selectively cleavable bond yields an Xpandomer of a length longer than the plurality of the XNTP subunits of the strand.

In at least one embodiment of the method for sequencing of the present disclosure, the method further comprises cleaving the selectively cleavable bonds to yield an Xpandomer.

In at least one embodiment of the method for sequencing of the present disclosure, the Xpandomer comprises reporter constructs for parsing genetic information in a sequence corresponding to the contiguous nucleotide sequence of all or a portion of the target nucleic acid. In at least one embodiment, the detecting changes in the voltage comprises detecting changes in the voltage due to the reporter constructs of the Xpandomer translocating the nanopore.

In at least one embodiment of the method for sequencing of the present disclosure, the reporter constructs for parsing the genetic information comprise a reporter code and a translocation control element, wherein the translocation control element provides translocation control by steric hindrance and pauses translocation of the Xpandomer when passed through a nanopore subjected to a baseline voltage, wherein the translocation control element engages the reporter code within the aperture of the nanopore, wherein the reporter code is sensed by the nanopore.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts an embodiment of a cell 100 in a nanopore based sequencing chip.

FIG. 2 depicts an embodiment of a cell 200 performing nucleotide sequencing with the Nano-SBS technique.

FIG. 3 depicts an embodiment of a cell about to perform nucleotide sequencing with pre-loaded tags.

FIG. 4 depicts an embodiment of a process 400 for nucleic acid sequencing with pre-loaded tags.

FIG. 5 depicts an embodiment of a circuitry 500 in a cell of a nanopore based sequencing chip.

FIG. 6 depicts an embodiment of a circuitry 600 in a cell of a nanopore based sequencing chip, wherein the voltage applied across the nanopore can be configured to vary over a time period during which the nanopore is in a particular detectable state.

FIG. 7 depicts a schematic illustration of features of a generalized XNTP and its function in a Nano-SBX technique.

FIG. 8 depicts a schematic illustration of features of a generalized XNTP and its function in a Nano-SBX technique.

FIG. 9 depicts a schematic illustration of features of a generalized XNTP and its function in a Nano-SBX technique.

FIG. 10 depicts a schematic illustration of features of a generalized XNTP and its function in a Nano-SBX technique.

FIG. 11 is a schematic illustrating more details of one embodiment of an XNTP.

FIG. 12 is a schematic illustrating one embodiment of an Xpandomer passing through a biological nanopore.

### DETAILED DESCRIPTION

The present disclosure provides electrochemical cells and associated buffer compositions for use in the cells. The electrochemical cells are useful for carrying out nanopore-based methods of assaying nucleic acids, including nanopore-based sequencing. The features of the electrochemical cells and associated buffer compositions result in the surprising, advantageous result of reducing formation of deleterious precipitants during the use of the cells in nanopore-based assays. The deleterious precipitants include Prussian Blue, which can precipitate as a solid in a redox reaction involving ferricyanide and ferrocyanide ions and metallic iron found in components of the electrochemical cell, such as the inlet and outlet ports. The formation of such precipitants can rapidly clog the ports and/or the nanopore thereby greatly decreasing the accuracy and efficiency of any nanopore-based assay. The present disclosure also provides, methods for using the electrochemical cells and compositions for nanopore-based nucleic acid detection techniques, such as nanopore-based sequencing-by-synthesis (Nano-SBS) and nanopore-based sequencing by expansion.

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50” includes “2 to 25”, “5 to 20”, “25 to 50”, “1 to 10”, etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present disclosure pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Nanopore-Based Devices for Detecting Nucleic Acids

Nanopore-based devices for detecting nucleic acids have been developed for rapid sequencing and various designs and methods of use are known in the art. See e.g., U.S. Pat. Nos. 9,494,554B2, 9,567,630B2, 9,557,294B2, 9,605,309B2, each of which hereby incorporated by reference herein. These devices generally comprise an electrochemical cell with a chamber containing a nanopore embedded in a membrane. The membrane acts to separate the cell chamber into two sub-chambers, referred to as the cis and trans sides of the cell, each of which contain an electrode. The membrane can be an organic membrane, such as a lipid bilayer, or a synthetic membrane made of a non-naturally occurring polymeric material. The pore of the nanopore acts as a channel (or passage) in the membrane between the cis and trans sides of the cell. Depending on the nanopore used, the pore has a width or diameter that can range from about 1 angstrom to about 10,000 angstroms. The nanopore can be a naturally-occurring pore-forming protein, such as α-hemolysin from S. aureus, non-naturally occurring mutant or variant of a wild-type pore-forming protein. A range of naturally and non-naturally occurring nanopores having varying pore-sizes and properties are known in the art. See e.g., U.S. Pat. Nos. 10,351,908B2, 10,934,582B2, 10,227,645B2.

Within the electrochemical cell, the nanopore embedded in the membrane is disposed in proximity to an electrode coupled to a sensing circuit, such as, for example, a complementary metal-oxide semiconductor (CMOS) or field effect transistor (FET) circuit. When a voltage potential is applied (via the electrodes) across a nanopore immersed in a conducting fluid, a small current attributed to the flow of ions through the nanopore can be observed. This ion flow is sensitive to the pore size, and thus, molecules entering the pore affect the ion flow and the voltage measured through this sensor circuit.

Electrochemical cells for nanopore-based sequencing of nucleic acids are typically used in a massively parallel fashion in which thousands of such cells are configured as an array in a single device often referred to as a chip (or bio-chip). A typically nanopore-based sequencing chip device incorporates an array of one million or more electrochemical cells, and may include 1000 rows by 1000 columns of such cells (see e.g., chips fabricated by Roche Sequencing Solutions, Santa Clara, CA, USA). Methods for fabricating and using such nanopore array microchips can also be found in U.S. Patent Application Publication Nos. 2013/0244340 A1, US 2013/0264207 A1, US2014/0134616 A1, 2015/0368710 A1, and 2018/0057870 A1, and published International Application WO 2019/166457 A1, each of which is hereby incorporated by reference herein. Each well in the array is manufactured using a standard CMOS process with surface modifications that allow for constant contact with biological reagents and conductive salts. Each well can support a phospholipid bilayer membrane with a nanopore-polymerase conjugate embedded therein. The electrode at each well is individually addressable by computer interface. All reagents used are introduced into a simple flow cell above the array microchip using a computer-controlled syringe pump. The chip supports analog to digital conversion and reports electrical measurements from all electrodes independently at a rate of over 1000 points per second. Nanopore measurements can be made asynchronously at each of 8 M addressable nanopore-containing membranes in the array at least once every millisecond (msec) and recorded on the interfaced computer. Further description of exemplary electrochemical cells useful for nanopore-based nucleic acid assays, such as sequencing, including chamber and electrode materials, buffer solutions, sensing circuitry, array devices, and their use in various applications is provided below.

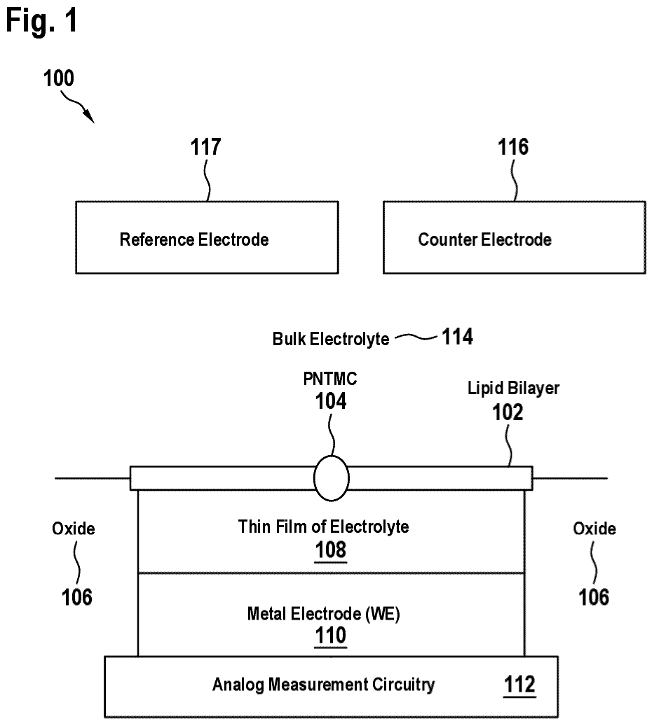

FIG. 1 illustrates an exemplary embodiment of an electrochemical cell 100 found in an array of such cells useful for nanopore-based sequencing. A membrane 102 is formed over a surface in the cell. In some embodiments, the membrane 102 is a lipid bilayer. A bulk electrolyte solution 114 containing the nanopore or “protein nanopore transmembrane molecular complex” (PNTMC) 104 is placed directly onto this surface in the cell and electroporation is used to insert a single PNTMC 104 into the membrane 102. The individual nanopore embedded membranes in each cell of an array are neither chemically nor electrically connected to each other. Thus, each cell is an independent sequencing machine, producing data unique to the single polymer molecule associated with the PNTMC.

With continued reference to FIG. 1, a thin film of electrolyte solution 108 is isolated from the bulk electrolyte solution 114 by the ion-impermeable membrane 102. The nanopore embedded membrane thus separates the electrochemical cell 100 into two chambers, a cis chamber containing the bulk electrolyte solution 114, and a trans chamber containing the thin film of electrolyte solution 108. The thin film of electrolyte solution 108 in the trans chamber is in contact with a metal working electrode 110 that is connected to analog measurement circuitry 112. The bulk electrolyte solution in the cis chamber is in contact with a counter electrode 116 and a reference electrode 117.

The pore of the PNTMC 104 provides a channel through the membrane 102 that allows analytes (e.g., nucleic acids) and ions to flow, which results in modulation of the ionic current across the impermeable bilayer. The pore of the PNTMC 104 thus provides the only path for ion current to flow from the metal working electrode 110 to the bulk electrolyte solution 114 in contact with the counter electrode 116. The electrochemical cell also includes a reference electrode 117, which further enhances it sensitivity as an electrochemical potential sensor.

The thin film of electrolyte constitutes a femtoliter volume of solution in the trans chamber that is in direct contact with the working electrode. This small volume of electrolyte contacting the electrodes of the electrochemical cell must undergo repeated voltage pulses in sensing ion flow through the nanopore. These repeated electrochemical measurements using electrodes in a small volume of electrolyte and the need to maintain ion flow through a nanopore with repeated measurements corresponding to individual molecular moieties translocating through the pore requires a sensor system of exquisite sensitivity. This electrochemical measurement system is highly sensitive to any precipitating or aggregating molecular entities that can clog the pore or otherwise interfere with the ionic flow between the working and counter electrode. Accordingly, the electrolyte salts, electrochemical cell materials, and electrochemical measurement conditions should be carefully controlled to prevent or reduce deleterious precipitant formation or electrode wear in the system.

The electrolyte solution 108 may include one of the following: lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl₂)), strontium chloride (SrCl₂), manganese chloride (MnCl₂), and magnesium chloride (MgCl₂). In some embodiments, the film of electrolyte solution has a thickness of about three microns (μm). The thickness of the film of electrolyte solution may range from 0-5 microns.

In addition to the salts described above, the electrolyte film can contain redox active ions such as ferrocyanide ion and ferricyanide ion. These redox ions are used in certain nanopore-based sequencing methods carried out in the electrochemical cell. For example, Nano-SBX measurements carried out in a cell of the present disclosure can comprise ferricyanide and ferrocyanide ions. Such redox active ions can undergo irreversible reactions depending on their concentration and the particular electrochemical conditions at the working and counter electrodes. Indeed, as described elsewhere herein, the irreversible formation of the precipitant Prussian Blue in an electrochemical cell is highly deleterious to any nanopore-based measurements in the cell. Accordingly, the concentration of such redox active ions as ferricyanide and ferrocyanide should carefully controlled. In at least one embodiment of the present disclosure, any ferricyanide and ferrocyanide ions in the solutions in the electrochemical cell should be maintained at a total concentration of between about 25 mM and 250 mM.

It has also been found that the salts used in electrolyte solutions of the electrochemical cell can affect the ability of the cell to be used in sensitive nanopore-based nucleic acid measurements. For example, many nanopore-based sequencing protocols use ammonium chloride as a buffer salt in the electrolyte solutions provided in the cell chambers. It has been found that controlling the concentration of ammonium chloride by substituting with ammonium acetate as a salt in the electrolyte solution during nanopore-based nucleic acid sequencing methods, such as Nano-SBX, can help reduce or prevent the formation of redox precipitants, such as Prussian Blue, which is, as mentioned above, highly deleterious to the accuracy and sensitivity of the electrochemical cell. Accordingly, in some embodiments of the electrochemical cell of the present disclosure, the buffer composition used in the cell comprises ammonium acetate at a concentration of from about 0 mM to about 1500 mM, and/or ammonium chloride at a concentration of less than 2000 mM. In at least one embodiment, the buffer composition comprises ammonium acetate at a concentration of from about 0 mM and 1500 mM and an ammonium chloride at a concentration of less than 2000 mM. In at least one embodiment, the concentration of ammonium acetate is 0 mM and the concentration of ammonium chloride is about 600 mM.

Dielectric materials are typically used to form oxide layers 106 that define the trans chamber of the cell. Useful dielectric materials can include glass, oxide, silicon mononitride (SiN), and the like. In some embodiments, the top surface of dielectric oxide layer 106 in that is in contact with bulk electrolyte 114 may be silanized. Silanization forms a hydrophobic layer above the top surface of dielectric layer. In some embodiments, this hydrophobic layer has a thickness of about 1.5 nanometer (nm). Alternatively, dielectric material that is hydrophobic such as hafnium oxide may be used to form the top of the dielectric layer.

As shown in FIG. 1, the membrane is a lipid bilayer formed at least partially on the dielectric oxide layer 106 and spans across the well containing the thin film of electrolyte 108. For example, the membrane can form on top of the hydrophobic layer on the dielectric oxide layer and as the membrane reaches the opening of well, the lipid monolayer transitions to a lipid bilayer that spans across the opening of the well. The hydrophobic layer can facilitate the formation of lipid monolayer above dielectric layer and the transition from a lipid monolayer to a lipid bilayer.

The bulk electrolyte 114 may further include one of the following: lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl₂)), strontium chloride (SrCl₂), manganese chloride (MnCl₂), and magnesium chloride (MgCl₂). Accordingly, in some embodiments of the electrochemical cell of the present disclosure, the buffer composition of the bulk electrolyte used in the cell comprises ammonium acetate at a concentration of from about 0 mM to about 1500 mM, and/or ammonium chloride at a concentration of less than 2000 mM. In at least one embodiment, the buffer composition comprises ammonium acetate at a concentration of from about 0 mM and 1500 mM and an ammonium chloride at a concentration of less than 2000 mM. In at least one embodiment, the concentration of ammonium acetate is 0 mM and the concentration of ammonium chloride is about 600 mM. Similarly, in some embodiments, any ferricyanide and ferrocyanide ions in the bulk electrolyte solution of the electrochemical cell should be maintained at a total concentration of between about 25 mM and 250 mM.

As noted above with respect to the thin electrolyte film, the composition of the salts (e.g., ammonium chloride versus ammonium acetate) and redox active ions used in the bulk electrolyte can affect the accuracy and sensitivity of the nanopore-based measurements made using the electrochemical cell. It is contemplated that depending on the type of electrochemical measurement being carried out, particularly the applied potentials across the electrodes in contact the solution, the composition of the solution can be adjusted to reduce or prevent deleterious precipitant formation.

In order to carry out the necessary insertion and removal of solutions, the electrochemical cells of the present disclosure include inlet and outlet ports operably connected to the chambers or reservoirs of the cell. The electrochemical cell schematic of FIG. 1 does not depict the inlet and outlet ports but the incorporation of such ports into electrochemical cells are well known in the art. The inlet and outlet ports allow the insertion of the reagents required for nanopore-based nucleic acid detection including the materials for creating the membrane with embedded nanopore, the nucleotide synthesis reagents, and the electrolyte and buffer solutions. Typically, the inlet and outlet ports comprise metal tubes fitted to the cell so as to provide a fluid connection from the outside into the chambers of the cell that can be used to insert or remove solutions. As such, at least a portion of the inlet and outlet port tubing is in contact with the electrochemical solutions in the cell chambers. Thus, the composition of the ports should be electrochemically inert at the voltages used to induce ion flow through the nanopore and make the sensory measurements of changes in ion flow as molecular moieties enter and/or translocate through the nanopore. Stainless steel tubing has been used to provide the metal tubing for inlet and outlet ports, however, steel contains iron. It is one surprising advantage of the electrochemical cells of the present disclosure that the inlet and outlet ports comprise a metal that contains less than 10% iron, and this in turn reduces or prevents deleterious formation of precipitants in the cell, such as Prussian Blue. Without intending to be bound by theory or mechanism, it is believed that corrosion of stainless steel components of electrochemical cells (e.g., ports) is accelerated by the presence of potassium ferricyanide (K₃[Fe(CN₆)₃]), which is a strong oxidizer, and chloride ions Cl, which induce pitting corrosion, causing the release of Fe³⁺ ions from stainless steel (and other iron-containing metals). The Fe³⁺ ions are capable of reacting with ferrocyanide ion ([Fe(CN)₆]⁴⁻) present in the solution resulting the formation of the precipitate Prussian Blue (Fe₄[Fe(CN)₆]₃). The solutions used in electrochemical cells for nanopore sequencing typically require the presence of ferrocyanide or ferricyanide ions, and it is believed that the stainless steel components (e.g., inlet and outlet ports) in contact with the solutions results in formation of the precipitant Prussian Blue under the electrochemical measurement conditions typically used in the cells for nanopore-based sequencing. The formation of the solid precipitant in the electrochemical cells results in clogs, and typically rapid failure of the cell during the course of the nanopore-based measurements.

Although it has been determined that iron-containing metals, such as stainless steel, are problematic for Prussian Blue formation, more specifically, it has been found that Prussian Blue or other metal precipitant formation can be reduced or prevented by using metal inlet and outlet ports wherein the metal exhibits a current density of less than or equal to 10⁻³ mA/cm² when polarized to a potential of about 0.3 V versus a Ag/AgCl reference electrode in a buffer solution of 1 M NH₄Cl, 800 mM urea, 100 mM HEPES, pH 7.4, or in a buffer solution of 1 M NH₄Cl, 800 mM urea, 100 mM MES at pH 6.2-6.8.

Accordingly, in at least one embodiment of the electrochemical cells of the present disclosure, the inlet and outlet ports of the cell are made of a metal that exhibits a current density of less than or equal to 10⁻³ mA/cm² when polarized to a potential of about 0.3 V versus a Ag/AgCl reference electrode in a buffer solution of 1 M NH₄Cl, 800 mM urea, 100 mM HEPES, pH 7.4, or in a buffer solution of 1 M NH₄Cl, 800 mM urea, 100 mM MES at pH 6.2-6.8. As noted above, generally, the metal should not contain iron, which excludes ports having stainless steel in contact with the solutions inside the cell during use. It has been found that ports made of nickel, such as a nickel alloy, that do not contain iron provide excellent reduction and/or prevention of Prussian Blue formation when the cell is used with solutions containing ferricyanide and ferrocyanide ions. In particular, inlet and outlet ports that are made of a nickel alloy selected from C276, C22, 625, and MP35N provide excellent nanopore-based sequencing measurements and lifetime.

The electrochemical cell 100 includes a counter electrode (CE) 116 and a reference electrode 117, which acts as an electrochemical potential sensor. In some embodiments, counter electrode 116 is shared between a plurality of cells, and is therefore also referred to as a common electrode. The common electrode can be configured to apply a common potential to the bulk liquid in contact with the nanopores in the plurality of cells. The common potential and the common electrode are common to all of the measurement cells.

In at least one embodiment, it has been found that a non-faradaic electrochemical cell with a titanium nitride (TiN) working electrode can be advantageous for nanopore-based sequencing of nucleic acids. The cell is similar in general structure to the cell of FIG. 1 but includes a TiN working electrode that exhibits increased electrochemical capacitance. The electrochemical cell further includes a conductive or metal layer that connects the working electrode of the cell to the circuitry of the array of which the cell is part.

Accordingly, in at least one embodiment, the metal working electrode 110 is a titanium nitride (TiN) metal electrode with increased electrochemical capacitance. The electrochemical capacitance associated with metal working electrode 110 may be increased by maximizing the specific surface area of the electrode. The specific surface area of the metal working electrode 110 is the total surface area of the electrode per unit of mass (e.g., m²/kg) or per unit of volume (e.g., m²/m³ or m⁻¹) or per unit of base area (e.g., m²/m²). As the surface area increases, the electrochemical capacitance of the metal working electrode increases, and a greater amount of ions can be displaced with the same applied potential before the capacitor becomes charged. The surface area of working electrode 110 may be increased by making the TiN electrode “spongy” or porous. The TiN sponge soaks up electrolyte and creates a large effective surface area in contact with the electrolyte.

The ratio of the capacitance associated with the membrane (C_{membrane}) and the capacitance associated with the working electrode (C_{electrochemical}) may be adjusted to achieve optimal overall system performance. Increased system performance may be achieved by reducing C_{membrane} while maximizing C_{electrochemical}. C_{membrane} is adjusted to create the required RC time constant without the need for additional on-chip capacitance, thereby allowing a significant reduction in cell size and chip size. The base surface area of the metal working electrode 110 is greater than or equal to the surface area of the opening of the trans side well containing the electrolyte 108 with walls defined by the oxide layers 106. Therefore, the two base surface areas may be optimized independently to provide the desired ratio between C_{membrane} and C_{electrochemical}. By using a spongy and porous TiN working electrode, the electrolyte can diffuse through the spaces between the columnar TiN structures and vertically down the uncovered portion of the working electrode and then horizontally to the covered portion of working electrode 1102 that is underneath dielectric layer 1104. As a result, the effective surface area of TiN that is in contact with the electrolyte is maximized and C_{electrochemical} is maximized.

Further description of the design of non-faradaic electrochemical cells, TiN working electrodes, and other nanopore-based cell designs useful with the devices, compositions, and methods of the present disclosure can be found in e.g., U.S. Pat. No. 10,174,371B2, which is hereby incorporated by reference herein.

As described elsewhere herein, the electrochemical cells of the present disclosure can be used as devices for nanopore-based nucleic acid detection and measurement assays, including nucleic acid sequencing. Generally, nanopore-based sequencing of a target nucleic acid can be carried using an electrochemical cell of design described above, and the associated solution compositions, including electrolyte and buffer solutions that reduce precipitant formation as described above. The method comprises: (a) providing an electrochemical cell comprising (i) a nanopore embedded in a membrane that separates the cell into cis and trans chambers operably connected by the nanopore, wherein the cis and trans chambers each contain an electrode, a solution comprising ferrocyanide ion, ferricyanide ion, and a buffer composition; and (ii) inlet and outlet ports comprising a metal operably connected to the cell; (b) adding a molecule derived from a target nucleic acid to the cis chamber; (c) applying a voltage to the cell that causes at least a portion of the molecule to translocate through the nanopore; and (d) detecting changes in voltage flow in the cell as the molecule translocates through the nanopore, wherein the changes in voltage flow are indicative of a sequence of the target nucleic acid.

As described elsewhere herein, the particular potentials applied, their timing and duration can have a significant effect on the highly sensitive changes in ion flow conductance through the nanopore that occurs in the presence of the nucleic acid molecular moieties being detected in carrying out a sequencing method. Exemplary methods and parameters for application of voltage potentials across nanopore-based electrochemical cell arrays for sequencing are known in the art and described in e.g., U.S. Pat. Nos. 11,150,216 and 11,029,306, each of which is hereby incorporated by reference herein The applied potentials, their pulse timing and duration can also affect the redox chemistry inside the cell resulting in the formation of deleterious precipitants such as Prussian Blue. It is a surprising advantage of the electrochemical cells and associated solutions used in them of the present disclosure that in some embodiments, a method of nanopore sequencing using an electrochemical cell of the present disclosure can be carried out using an alternating current (AC) applied to the cell with a voltage range of 450 mV to 1200 mV for 10 hours resulting in no visible formation of Prussian Blue in the cell.

In some embodiments of the method for sequencing of the present disclosure, the applied voltage comprises applying a baseline voltage potential to the cell. A typical baseline voltage applied can be in the range of from about 55 mV to about 95 mV.

In some embodiments of the methods for sequencing, the applied voltage can include a pulse voltage. Pulse voltages useful in the method can be from about 320 mV to about 550 mV. The duration of the pulse voltage typically is from about 5 μs to about 10 μs. In at least one embodiment, the time between pulse voltages is about 0.5 ms to 1.7 ms. In at least one embodiment, the applied voltage provides an alternating current to the electrochemical cell. For example, the resulting alternating current can have a periodicity of about 0.4 s to about 6 s.

In at least one embodiment, it is contemplated that an array of electrochemical cells of the present disclosure can be used to carry out a method of sequencing in a parallel fashion. Such parallel nanopore-based sequencing of nucleic acids using parallel arrays of electrochemical cells have been used with the nanopore-based sequencing by synthesis (Nano-SBS) technique. Systems compositions, and methods for Nano-SBS are known in the art. See e.g., US Pat. Publ. Nos. 2013/0244340 A1, 2013/0264207 A1, 2014/0134616 A1, 2015/0368710 A1, and 2018/0057870 A1, and published International Application WO 2019/166457 A1. It is contemplated that these known systems, compositions and methods can be used or adapted for use with the electrochemical cells of the present disclosure. A description of the use Nano-SBS in the context of the electrochemical cells and compositions of the present disclosure is provided below.

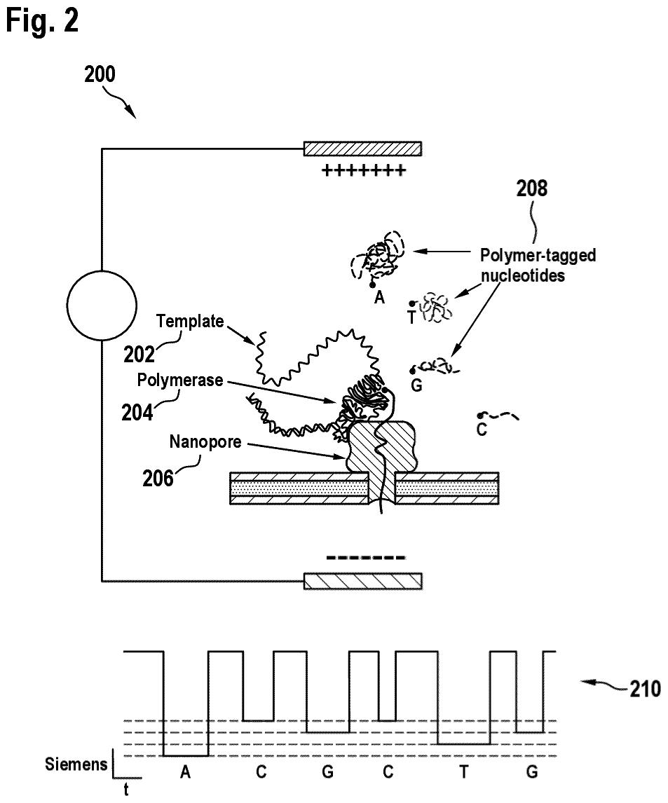

FIG. 2 illustrates an embodiment of a electrochemical cell 200 used to carry out nanopore-based nucleic acid sequencing with the Nano-SBS technique. In the Nano-SBS technique, a template 202 to be sequenced and a primer are introduced into an electrochemical cell 200. To this template-primer complex, four differently tagged nucleotides 208 are added to the bulk aqueous phase. As the correctly tagged nucleotide is complexed with the polymerase 204, the tail of the tag is positioned in the barrel of nanopore 206. The tag held in the barrel of nanopore 206 generates a unique ionic blockade signal 210, thereby electronically identifying the added base due to the tags' distinct chemical structures.

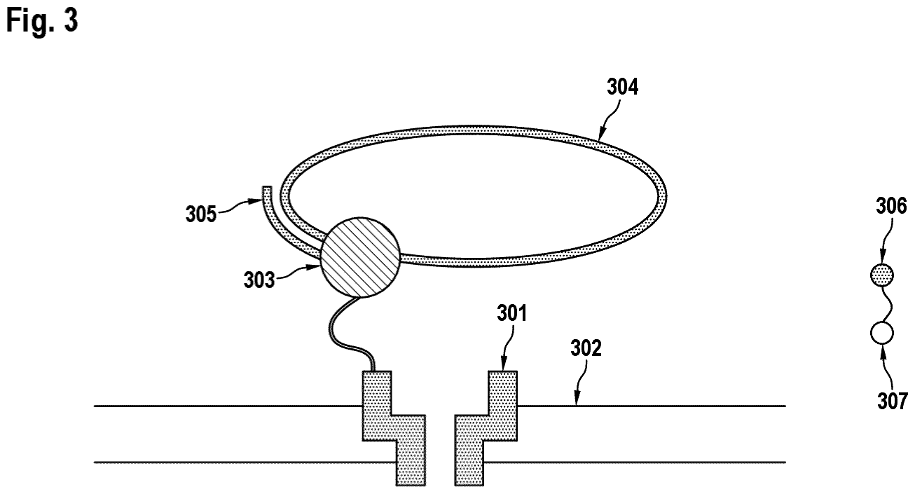

FIG. 3 illustrates an embodiment of an cell about to perform nucleotide sequencing with pre-loaded tags. A nanopore 301 is formed in a membrane 302. An enzyme 303 (e.g., a polymerase, such as a DNA polymerase) is associated with the nanopore. In some cases, the enzyme 303 is covalently attached to nanopore 301. Polymerase 303 is associated with a nucleic acid molecule 304 to be sequenced. The associated nucleic acid molecule 304 can be linear or circular. In some embodiments, a nucleic acid primer 305 is hybridized to a portion of nucleic acid molecule 304. The polymerase 303 catalyzes the incorporation of nucleotides 306 onto primer 305 using single stranded nucleic acid molecule 304 as a template. As noted above, the nucleotides 306 comprise a tag species (“tags”) 307 that allows it to be distinguished from the other three nucleotides.

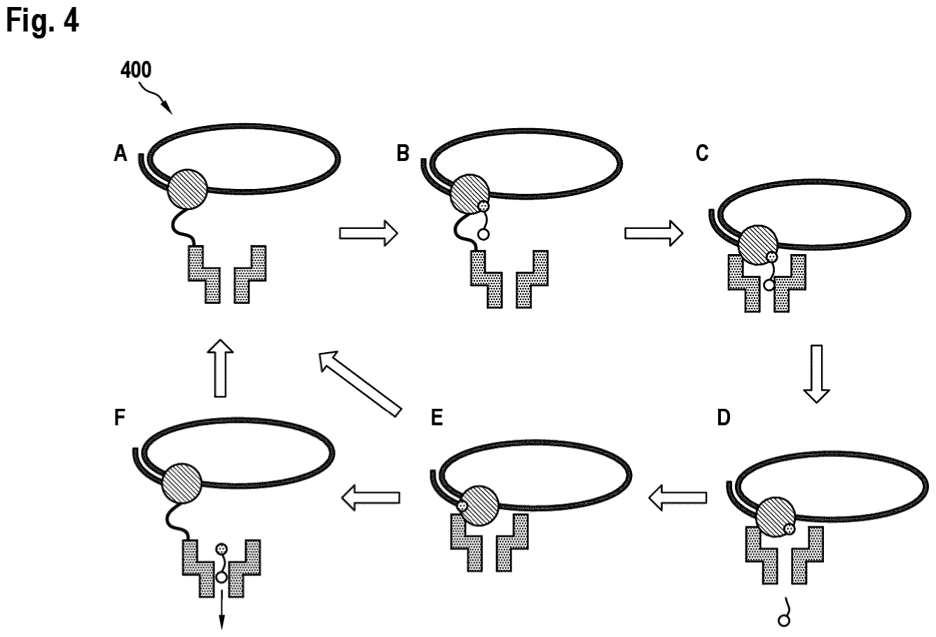

FIG. 4 illustrates an embodiment of a process 400 for nucleic acid sequencing with pre-loaded tags. At stage A, a tagged nucleotide (one of four different types: A, T, G, or C) is not associated with the polymerase. At stage B, a tagged nucleotide is associated with the polymerase. At stage C, the polymerase is in close proximity to the nanopore. The tag is pulled into the nanopore by an electrical field generated by a voltage applied across the membrane and/or the nanopore. Some of the associated tagged nucleotides are not base paired with the nucleic acid molecule. These non-paired nucleotides typically are rejected by the polymerase within a time scale that is shorter than the time scale for which correctly paired nucleotides remain associated with the polymerase. Since the non-paired nucleotides are only transiently associated with the polymerase, process 400 as shown in FIG. 4 typically does not proceed beyond stage B.

Before the polymerase is docked to the nanopore, the conductance of the nanopore is ˜300 pico Siemens (300 pS). At stage C, the conductance of the nanopore is about 60 pS, 80 pS, 100 pS, or 120 pS corresponding to one of the four types of tagged nucleotides. The polymerase undergoes an isomerization and a transphosphorylation reaction to incorporate the nucleotide into the growing nucleic acid molecule and release the tag molecule. In particular, as the tag is held in the nanopore, a unique conductance signal (e.g., see signal 210 in FIG. 2) is generated due to the tag's distinct chemical structures, thereby identifying the added base electronically. Repeating the cycle (i.e., stage A through E or stage A through F) allows for the sequencing of the nucleic acid molecule. At stage D, the released tag passes through the nanopore.

In some cases, tagged nucleotides that are not incorporated into the growing nucleic acid molecule will also pass through the nanopore, as seen in stage F of FIG. 4. The unincorporated nucleotide can be detected by the nanopore in some instances, but the method provides a means for distinguishing between an incorporated nucleotide and an unincorporated nucleotide based at least in part on the time for which the nucleotide is detected in the nanopore. Tags bound to unincorporated nucleotides pass through the nanopore quickly and are detected for a short period of time (e.g., less than 10 ms), while tags bound to incorporated nucleotides are loaded into the nanopore and detected for a long period of time (e.g., at least 10 ms).

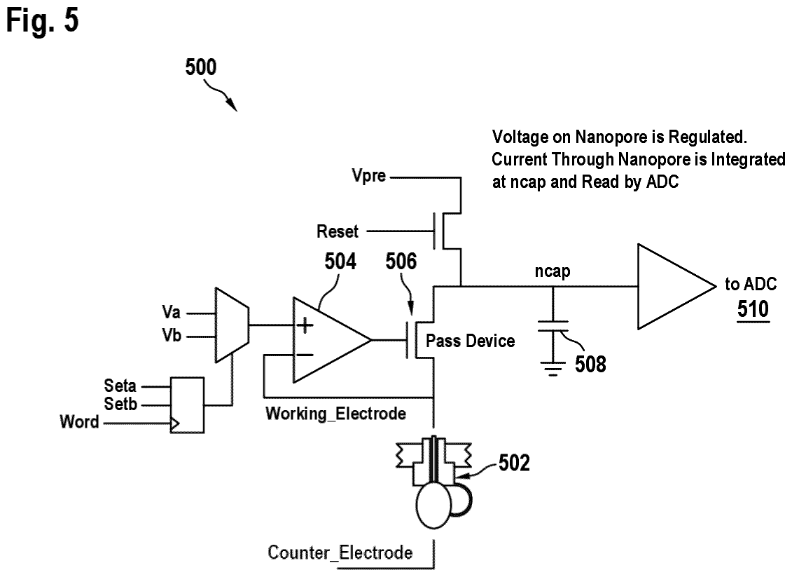

FIG. 5 illustrates an embodiment of a circuitry 500 in an electrochemical cell of a nanopore-based sequencing chip. As mentioned above, when a molecular moiety (e.g., a tag) enters nanopore 502, a unique conductance signal (e.g., see signal 210 in FIG. 2) is generated due to the distinct chemical structure and how it fits in the nanopore, thereby identifying the added base electronically. The circuitry in FIG. 5 maintains a constant voltage across nanopore 502 while the ion flow conductance is measured. In particular, the circuitry includes an operational amplifier 504 and a pass device 506 that maintain a constant voltage equal to Vₐ or V_{b} across the nanopore 502. The ion flow through nanopore 502 is integrated at a capacitor n_{cap} 508 and measured by an Analog-to-Digital (ADC) converter 510. The circuitry 500, however, only measures unidirectional current flow. Additionally, the temperature drift in the operational amplifier 504 may cause the actual voltage applied across nanopore 502 to vary across different electrochemical cells in an array. The actual voltage applied across nanopore 502 may drift by tens of millivolts above or below the desired value, thereby causing significant measurement inaccuracies. Shrinking the operational amplifier's size in a large-sized array may raise other performance issues.

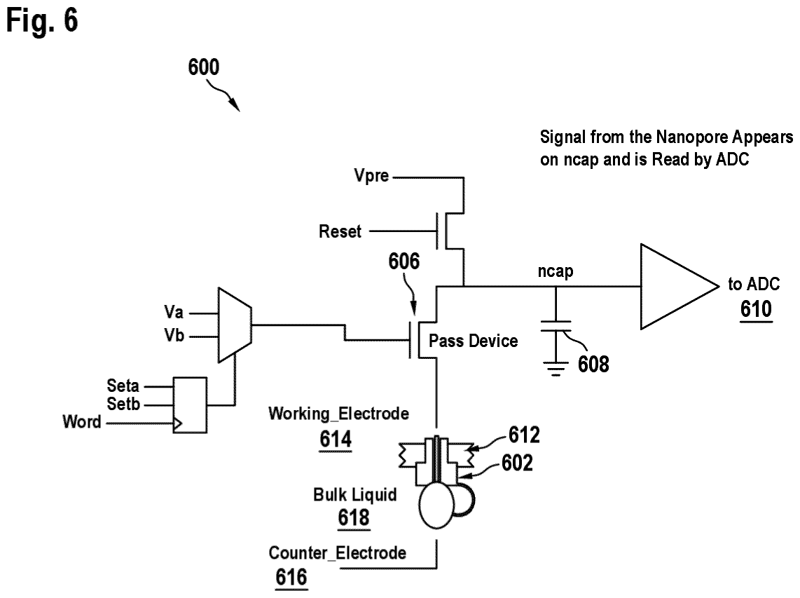

FIG. 6 illustrates an embodiment of circuitry 600 for use in an electrochemical cell of a nanopore based sequencing chip, wherein the voltage applied across the nanopore can be configured to vary over a time period during which the nanopore is in a particular detectable state. One of the possible states of the nanopore is an open-channel state when a no molecular moiety (e.g., a tag) is present in the barrel of the nanopore. Other possible states of the nanopore correspond to the states when the four different types of molecular moieties are present in the barrel of the nanopore. Yet another possible state of the nanopore is when the membrane is ruptured. FIG. 6 shows a nanopore 602 that is inserted into a membrane 612, and nanopore 602 and membrane 612 are situated between a cell working electrode 614 and a counter electrode 616, such that a voltage is applied across nanopore 602. Nanopore 602 is also in contact with a bulk liquid/electrolyte 618. Note that nanopore 602 and membrane 612 are drawn upside down as compared to the nanopore and membrane in FIG. 1. Hereinafter, an electrochemical cell is meant to include at least a membrane, a nanopore, a working electrode, and the associated circuitry. In some embodiments, the counter electrode is shared between a plurality of cells (e.g., in an array), and is therefore also referred to as a common electrode. The common electrode can be configured to apply a common potential to the bulk liquid in contact with the nanopores in the measurement cells. The common potential and the common electrode are common to all of the measurement cells. There is a metal working cell electrode within each measurement cell; in contrast to the common electrode, the metal working cell electrode 614 is configurable to apply a distinct potential that is independent from the working cell electrodes in other measurement cells.

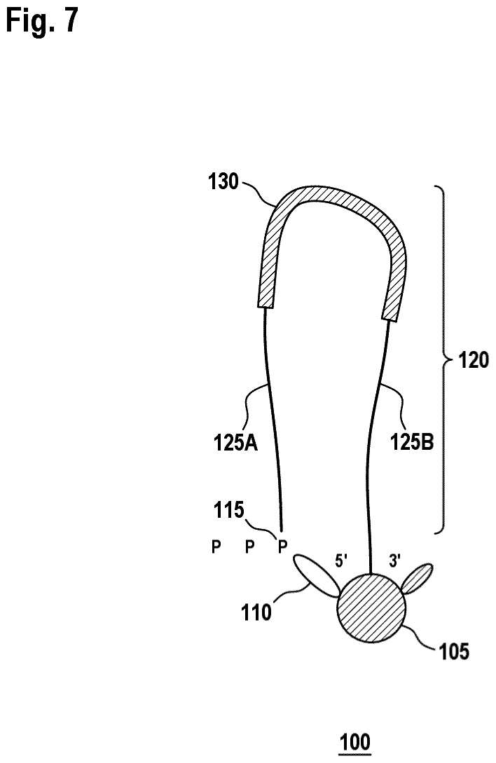

In addition to the Nano-SBS sequencing described above, in some embodiments, the electrochemical cells of the present disclosure can be used to carry out parallel sequencing of nucleic acids using a nanopore-based Sequencing-by-Expansion (Nano-SBX) technique. See, e.g., U.S. Pat. No. 7,939,259. The SBX technique is based on the polymerization of highly modified, non-natural nucleotide analogs referred to as “XNTPs”. In general terms, SBX uses biochemical polymerization to transcribe the sequence of a DNA template onto a measurable polymer called an “Xpandomer”. The transcribed sequence is encoded along the Xpandomer backbone in high signal-to-noise reporters that are separated by ˜10 nm and are designed for high-signal-to-noise, well-differentiated responses. These differences provide significant performance enhancements in sequence read efficiency and accuracy of Xpandomers relative to natural DNA. A general description of the SBX process is depicted in FIGS. 7, 8, 9, and 10.

XNTPs are expandable, 5′ triphosphate modified non-natural nucleotide analogs compatible with template dependent enzymatic polymerization. A highly simplified XNTP is illustrated in FIG. 7, which emphasizes the unique features of these non-natural substrates: XNTP 100 has two distinct functional regions; namely, a selectively cleavable phosphoramidate bond 110, linking 5′ α-phosphate 115 to the nucleobase 105, and a symmetrically synthesized reporter tether (SSRT) 120 that is attached within the nucleoside triphosphoramidate at positions that allow for controlled expansion by cleavage of the phosphoramidate bond. The SSRT includes linkers 125A and 125B separated by the selectively cleavable phosphoramidate bond. Each linker attaches to one end of a reporter code 130. XNTP 100 is illustrated in the “constrained configuration,” characteristic of the XNTP substrates and the daughter strand products of template-dependent polymerization. The constrained configuration of polymerized XNTPs is the precursor to the expanded configuration, as found in Xpandomer products. The transition from the constrained configuration to the expanded configuration occurs upon scission of the P—N bond of the phosphoramidate within the primary backbone of the daughter strand.

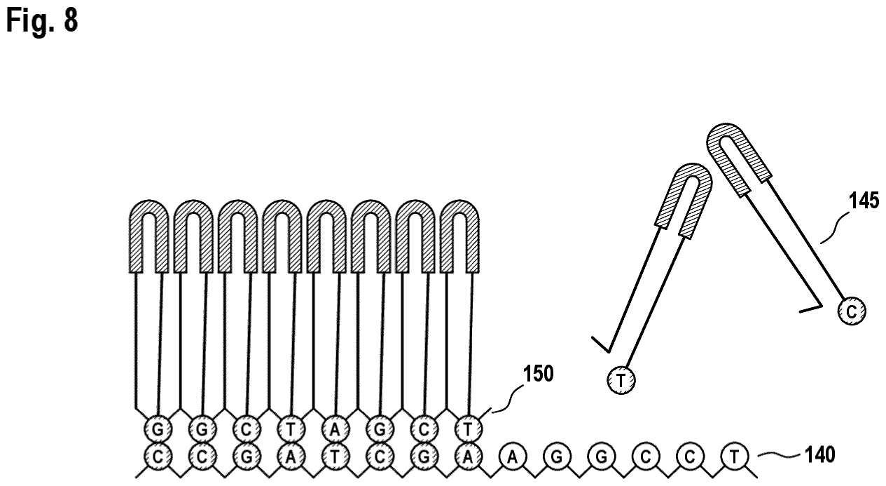

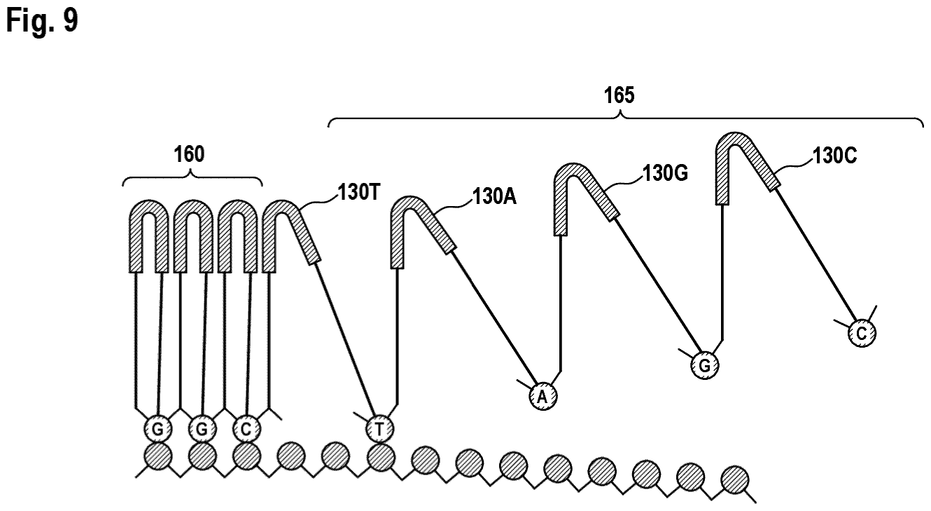

Synthesis of an Xpandomer polymer is summarized in FIGS. 8 and 9. As shown in FIG. 8, during assembly, the monomeric XNTP substrates 145 (XATP, XCTP, XGTP and XTTP) are polymerized on the extendable terminus of a nascent daughter strand 150 by a process of template-directed polymerization using single-stranded template 140 as a guide. Generally, this process is initiated from a primer and proceeds in 5′ to 3′ direction. Generally, a DNA polymerase or other polymerase is used to form the daughter strand, and conditions are selected so that a complimentary copy of the template strand is obtained. After the daughter strand is synthesized, the coupled SSRTs form the constrained Xpandomer that further forms the daughter strand. SSRTs in the daughter strand have the “constrained configuration” of the XNTP substrates. The constrained configuration of the SSRT is the precursor to the expanded configuration, as found the Xpandomer product.

As shown in FIG. 9, the transition from the constrained configuration 160 to the expanded configuration 165 results from cleavage of the selectively cleavable phosphoramidate bonds (illustrated for simplicity by the unshaded ovals) within the primary backbone of the daughter strand. In this embodiment, the SSRTs include one or more reporters or reporter codes, 130A, 130C, 130G, or 130T, specific for the nucleobase to which they are linked, thereby encoding the sequence information of the template. In this manner, the SSRTs provide a means to expand the length of the Xpandomer and lower the linear density of the sequence information of the parent strand.

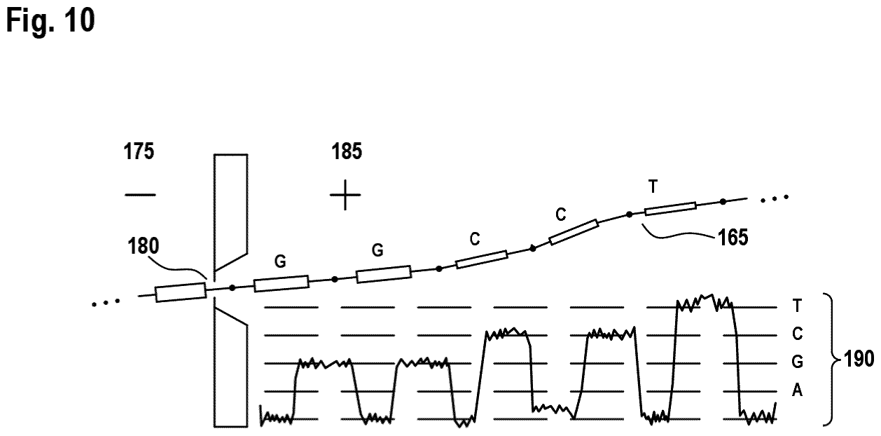

FIG. 10 illustrates an Xpandomer 165 translocating through a nanopore 180, from the cis chamber 175 to the trans chamber 185. Upon passage through the nanopore, each of the reporter codes of the linearized Xpandomer generates a distinct and reproducible electronic signal specific for the nucleobase to which it is linked. This signal is illustrated by superimposed trace 190.

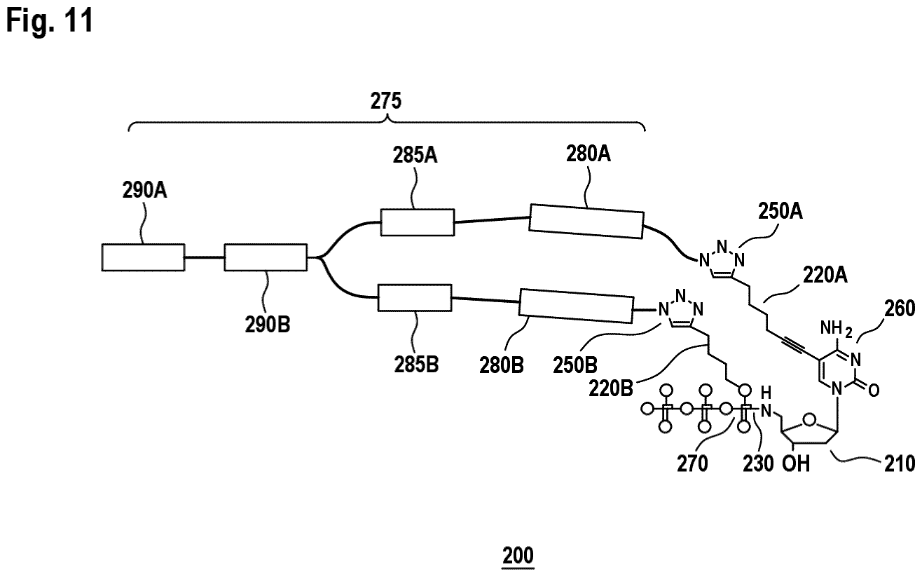

FIG. 11 depicts the generalized structure of one embodiment of an XNTP in more detail. XNTP 200 includes nucleoside triphosphoramidate 210 with linker arm moieties 220A and 220B separated by selectively cleavable phosphoramidate bond 230. An SSRT 275 is joined to the nucleoside triphosphoramidate at linkage groups 250A and 250B, in which a first SSRT end is joined to the heterocycle 260 (represented here by cytosine, though the heterocycle may be any one of the four standard nucleobases, A, C, G, or T) and a second SSRT end is joined to the α-phosphate 270 of the nucleobase backbone. The skilled artisan will appreciate that many suitable coupling chemistries known in the art may be used to form the final XNTP substrate product, for example, SSRT conjugation may be accomplished through formation of a triazole linkage group.

In this embodiment, SSRT 275 includes several functional elements, or “features” such as polymerase enhancement regions 280A and 280B, reporter codes 285A and 285B, and translation control element (TCEs) 290A and 290B. In other embodiments, the SSRT can include a single TCE. Each of these features performs a unique function during translocation of the Xpandomer through a nanopore to produce a series of unique and reproducible electronic signal. SSRT 275 is designed for controlling the rate of Xpandomer translocation by the TCE through a combination of steric hindrance and/or electro-repulsion, as discussed elsewhere herein. Different reporter codes are sized to block ion flow through a nanopore at different measurable levels.

Specific SSRT polymeric sequences can be efficiently synthesized using phosphoramidite chemistry typically used for oligonucleotide synthesis. Reporter codes and other features can be designed by selecting a sequence of specific phosphoramidites from commercially available and/or proprietary libraries. Such libraries include, but are not limited to, polyethylene glycol with lengths of 1 to 12 or more ethylene glycol units and aliphatic polymers with lengths of 1 to 12 or more carbon units. In certain embodiments, the SSRTs include features referred to as “polymerase enhancement regions” at the ends of the SSRTs proximal to the nucleotide triphosphoramidate diester. Polymerase enhancement regions may include positively charged polyamine spacers (e.g., primary, secondary, tertiary, or quarternary amines) or triamine spacers (three secondary amines each separated by three carbons) that facilitate incorporation of XNTP structures by a nucleic acid polymerase. In certain embodiments, the polymerase enhancement region includes two repeat units of spermine, in which the spermine moiety is provided by a phosphoramidite monomer having the following structure (as one of skill in the art will recognize, the trifluoroacetamide protecting groups are removed at the end of SSRT synthesis to expose the amine groups on spermine):

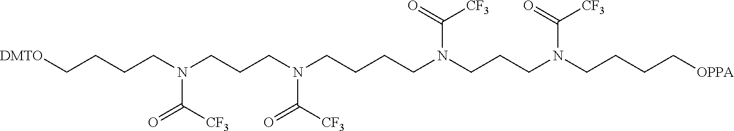

As used throughout the present disclosure, the term “reporter construct” refers to the element of the SSRT that includes the reporter codes, a symmetrical chemical brancher, and a translocation control element. In certain embodiments, the reporter construct is a polymer that includes, in series, from a first end to a second end, a first reporter code, a symmetrical chemical brancher bearing a translocation control element, and a second reporter code. The term “bearing” refers to a covalent linkage between the symmetrical brancher and the translocation control element, which produces an advantageous orientation of the translocation control element with respect to the two reporter codes. The symmetrical chemical brancher can be represented by the letter “Y”, in which the two reporter codes are joined to the arms of the Y, while the translocation control element is joined to the stem of the Y. Thus, the two reporter codes are joined in-line by the brancher, while the brancher bears the translocation control element in a perpendicular orientation with respect to the linear, in-line, SSRT.

As used throughout the present disclosure, the terms “linker A” and “linker B” refer to the regions of the SSRT that each include a polymerase enhancing region and one or more translocation deceleration features or regions, and, in certain embodiments, a spacer region that includes a polymer of, e.g., PEG6, which can be customized to modulate the length of the SSRT traversed in a nanopore.

In certain embodiments, an XNTP may be a compound having the following generalized structure:

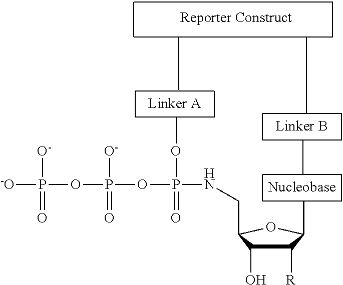

In one embodiment, R may be H, for example, when the compounds are used to sequence a DNA template. In another embodiment, R may be OH, for example, when the compounds are used to sequence an RNA template. In certain embodiments, nucleobase is adenine, cytosine, guanine, thymine, uracil or a nucleobase analog. As one of skill in the art will appreciate, adenine, cytosine, guanine, thymine, and uracil are naturally occurring nucleobases. As used herein, the term “nucleobase analog” refers to non-naturally occurring nucleobases that are capable of forming Watson and Crick base pair with a complementary nucleobase on an adjacent single-stranded nucleic acid template. Exemplary nucleobase analogs include, but are not limited to, 5-fluorouracil; 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, 8-aza-7-deazaguanine, 8-aza-7-deazainosine, and 8-aza-7-deazaadenine.

As discussed elsewhere herein, the reporter construct is a polymer having a first end and a second end, and includes, in series from the first end to the second end, the first reporter code, the symmetrical chemical brancher bearing the translocation control element, and the second reporter code. This series of features reflects the symmetrical structure of the reporter construct (and the entire SSRT, which includes the symmetrical linkers, linker A and linker B), in which the sequences of the two reporter codes are identical and joined, in-line in reverse orientation by the symmetrical chemical brancher. Briefly, synthesis proceeds in the 3′ to 5′ direction, initiating at 3′ end of the TCE. Addition of the symmetrical brancher to 5′ end of the TCE enables simultaneous polymerization of the first and second reporter codes off each arm of the brancher, followed by simultaneous synthesis of linker A and linker B, terminating at the 5′ end of the first end and the second end of the SSRT. It has been found that the in-line redundancy provided by two identical reporter codes separated by the symmetrical brancher bearing the translocation control element offers several advantages during nanopore sequencing. For example, Xpandomers can potentially be read by the nanopore when translocated in either direction, i.e., the Xpandomer can be read either “forwards” or “backwards”. This flexibility enables the “ratcheting” method of sequencing, which is discussed further herein, and other methods, such as “flossing” that are based on an AC pattern of voltage application.

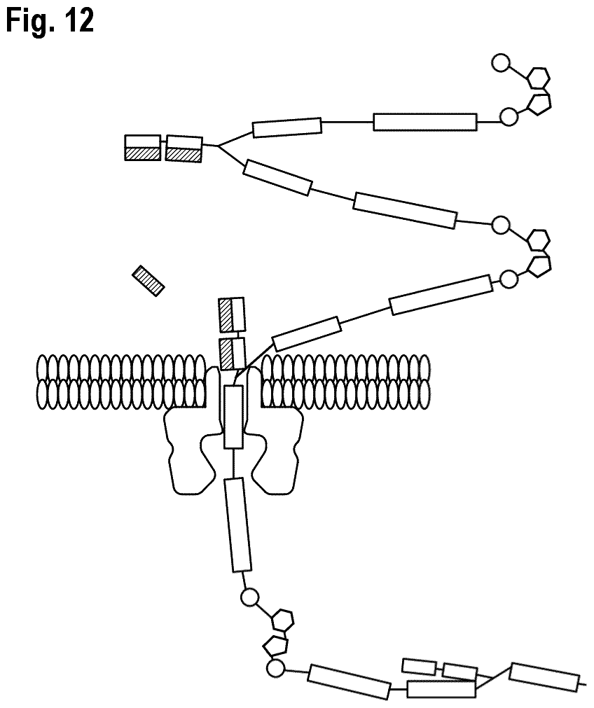

FIG. 12 shows one embodiment of a cleaved Xpandomer in the process of translocating a nanopore. The nanopore is embedded into a lipid bilayer membrane which separates and electrically isolates two chambers of electrolytes. A typical electrolyte has 1 M KCl buffered to a pH of 7.0. When a small voltage, typically 100 mV, is applied across the bilayer, the nanopore provide the only channel for the flow of ions induced by the potential and is the source primary resistance in the circuit. Xpandomer reporter codes are designed to give specific ion flow blockage levels and sequence information can be read by measuring the sequence of ion flow levels as pulses in voltage potential result in a sequence of reporter codes translocating through the nanopore. In the case of an α-hemolysin nanopore, the nanopore is typically embedded in the membrane so that translocation occurs by entering the cis vestibule side and exiting the trans stem side. As shown in FIG. 12, the nanopore is oriented to capture the Xpandomer from the stem side first. As the Xpandomer translocates the nanopore, a reporter enters the stem until its translocation control element stops at the stem entrance. The reporter is held in the stem until the TCE is enabled to pass into and through the stem, whereupon translocation proceeds to the next reporter. In this embodiment, TCE passage into the stem is enabled by dissociation of a translocation control moiety from the TCE.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Effect of Alternative Buffer Components and Metal Materials on Prussian Blue Formation

This example illustrates a study of the effect buffer components and metal surface compositions used in electrochemical cells on the formation of Prussian Blue.

### Materials and Methods

The standard femtoliter range chambers of an electrochemical cell used for SBX nanopore sequencing has interior stainless steel surfaces, including inlet and outlet ports, in contact with solutions contained in the cell that include ammonium chloride and approximately 250 mM each of potassium ferrocyanide and potassium ferricyanide. In order to simulate the conditions that occur in such a femtoliter nanopore sequencing chamber at a higher volume, a scratched cannula, made of varying types of metal alloy, was immersed in a buffer containing either ammonium chloride or ammonium acetate, varying amounts of potassium ferrocyanide and potassium ferricyanide, and various other buffering species, such as sodium salicylate, sodium pyrophosphate, resorcinol, and sulfosalicylic acid. The cannula was monitored for formation of Prussian Blue precipitate on the surface. The varying conditions and results of the study are summarized in Table 1 below.

TABLE 1

| Change to buffer/cannula | Prussian Blue formation? | Conclusion |
|---|---|---|
| None | Almost immediate formation of | Not |
| | Prussian Blue on scratched SS316 | effective |
| Halving of FeCN species concentration | Negligible formation of Prussian | Effective |
| to 125 mM | Blue on scratched surface, even | |
| | after multiple days | |
| Use of ammonium acetate (1M) instead | No formation of Prussian Blue on | Highly |
| of ammonium chloride | scratched surface, even after weeks | effective |
| Addition of 50 mM sodium salicylate | Initial formation of Prussian Blue | Somewhat |
| (pH ~7.4) to standard buffer | on scrached surface, but after | effective |
| | approximately 15 min complete | |
| | redissolution of material | |
| Use of C276 material instead of SS316 | No formation of Prussian Blue on | Highly |
| | scratched surface, even after weeks | effective |
| Use of C276 material and ammonium | No formation of Prussian Blue on | Highly |
| acetate | scratched surface, even after weeks | effective |
| Addition of 25 mM sodium | Slow formation of Prussian Blue on | Ineffective |
| pyrophosphate to buffer | scratched SS316 | |
| 45% Nitric acid pretreatment of SS316 | Slow formation of Prussian Blue on | Effective |
| | scratched SS316 (days) | |
| Addition of 50 mM resorcinol to | Almost immediate formation of | Ineffective |
| standard buffer | Prussian Blue on scratched SS316 | |
| Addition of 50 mM sulfosalicylic acid | Slow formation of Prussian Blue on | Ineffective |
| to standard buffer | scrached SS316 | |
| Addition of 100 mM sodium | Very slow formation of Prussian | Somewhat |
| pyrophosphate to buffer | Blue on strached SS316 | effective |
| Use of C22 material instead of SS316 | Negligible formation of Prussian | Effective |
| | Blue on scratched surface, even | |
| | after multiple days | |
| Use of gold coated material instead of | Rapid formation of a different color | Ineffective |
| SS316 | precipitate | |
| Use of C276 material and halving of | No formation of Prussian Blue on | Highly |
| FeCN species concentration to 125 mM | scratched surface, even after weeks | effective |

As shown by the results summarized in Table 1, the replacement of the stainless steel SS316 cannula with the non-iron containing nickel alloy C276 or C22 cannulas was highly effective in preventing formation of Prussian Blue over the course of days or even weeks.

Additionally, changing the buffer components had a large effect on Prussian blue formation. Specifically, replacement of ammonium chloride with ammonium acetate was highly effective in preventing Prussian Blue formation. Similarly, reducing the levels of the ferrocyanide and ferricyanide species by half greatly reduced Prussian Blue formation.

### Example 2: Effect of Alternative Buffer Components and Metal Materials on Prussian Blue Formation During Nanopore Sequencing

This example illustrates a study of the effect of buffer components and metal surfaces on formation of Prussian Blue in electrochemical cells during SBX nanopore sequencing.

As described elsewhere herein, SBX nanopore sequencing are carried out using an array of electrochemical cells containing a buffer solution containing ferricyanide and ferrocyanide ions. Solutions are flowed into and out of the cells during use via inlet and outlet ports.

Materials and methods: A series of hundreds of SBX nanopore-based sequencing experiments were carried out over the course of several months using standard arrays nanopore containing electrochemical cells with inlet and outlet ports made of either stainless steel (316 stainless) or a nickel alloy (alloys C276, C22, or 625) to flow the SBX sequencing buffer solutions through the cells. Cells in the arrays were monitored for failure, and the failed cells were analyzed for the presence of Prussian Blue precipitant clogs in the ports.

Results: It was found that cell failures due to Prussian Blue precipitant clogs were reduced from ˜35% to less than 5% with the change in port material from stainless steel to a nickel alloy.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. An electrochemical cell comprising:
(a) a nanopore embedded in a membrane that separates the cell into cis and trans chambers connected by the nanopore, wherein the cis and trans chambers each contain an electrode, a solution comprising ferrocyanide ion, ferricyanide ion, and a buffer composition; and
(b) inlet and an outlet ports operably connected to the cell, wherein the ports comprise a metal, wherein the metal exhibits a current density of Less than or equal to 10⁻³ mA/cm² when polarized to a potential of about 0.3 V versus a Ag/AgCl reference electrode in a buffer solution of 1 M NH₄Cl, 800 mM urea, 100 mM HEPES, pH 7.4, or in a buffer solution of 1 M NH₄Cl, 800 mM urea, 100 mM MES at pH 6.2-6.8.

2. (canceled)

3. The cell of claim 1, wherein the metal contains less than 10% iron, less than 8% iron, less than 6% iron, or less than 5% iron.

4. The cell of claim 1, wherein the metal is a nickel alloy.

5. (canceled)

6. The cell of claim 1, wherein the cell encloses a solution volume of between about 0.1 and about 1000 femtoliters.

7. The cell of claim 1, wherein the ferrocyanide and ferricyanide ions are at a total concentration of between about 25 mM and 250 mM.

8. The cell of claim 1, wherein the buffer composition comprises one or more of the following components: ammonium acetate at a concentration of from about 0 mM to about 1500 mM; and ammonium chloride at a concentration of less than 2000 mM, or less than 1000 mM.

9. (canceled)

10. (canceled)

11. The cell of claim 1, wherein the application of an alternating current (AC) to the cell with a voltage range of 450 mV to 1200 mV resulting in no visible formation of Prussian Blue in the cell for at least 1 hour, at least 2 hours, at least 4 hours, at least 6 hours, at least 8 hours, or at least 10 hours.

12. A method for sequencing a target nucleic acid comprising:
(a) providing an electrochemical cell comprising (i) a nanopore embedded in a membrane that separates the cell into cis and trans chambers operably connected by the nanopore, wherein the cis and trans chambers each contain an electrode, a solution comprising ferrocyanide and ferricyanide ions, and a buffer composition; and (ii) inlet and outlet ports comprising a metal connected to the cell;
(b) adding a molecule derived from a target nucleic acid to the cis chamber;
(c) applying a voltage to the cell that causes at least a portion of the molecule to translocate through the nanopore; and
(d) detecting changes in voltage flow in the cell as the molecule translocates through the nanopore, wherein the changes in voltage flow are indicative of a sequence of the target nucleic acid,
wherein the metal exhibits a current density of less than or equal to 10⁻³ mA/cm² when polarized to a potential of about 0.3 V versus a Ag/AgCl reference electrode in a buffer solution of 1 M NH₄Cl, 800 mM urea, 100 mM HEPES, pH 7.4, or in a buffer solution of 1 M NH₄Cl, 800 mM urea, 100 mM MES at pH 6.2-6.8.

13. (canceled)

14. The method of claim 12, wherein the metal contains less than 10% iron, less than 8% iron, less than 6% iron, or less than 5% iron.

15. (canceled)

16. (canceled)

17. (canceled)

18. (canceled)

19. (canceled)

20. (canceled)

21. The method of claim 12, wherein the application of an alternating current (AC) to the cell with a voltage range of 450 mV to 1200 mV resulting in no visible formation of Prussian Blue in the cell for at least 1 hour, at least 2 hours, at least 4 hours, at least 6 hours, at least 8 hours, or at least 10 hours.

22. The method of claim 12, wherein the applied voltage comprises a baseline voltage from about 55 mV to about 95 mV.

23. The method of claim 12, wherein the applied voltage comprises a pulse voltage from about 320 mV to about 550 mV.

24. The method of claim 12, wherein the pulse voltage has a duration from about 5 μs to about 15 μs.

25. The method of claim 12, wherein the time between pulse voltages is from about 0.5 ms to about 1.7 ms.

26. The method of claim 12, wherein the applied voltage provides an alternating current having a periodicity of about 0.4 s to about 6 s.

27. The method of claim 12, wherein the molecule derived from the target nucleic acid comprises an Xpandomer including a plurality of XNTP subunits coupled in a sequence corresponding to a contiguous nucleotide sequence of all or a portion of the target nucleic acid, wherein the individual XNTP subunits of the strand comprise a reporter construct, a nucleobase residue, and a selectively cleavable bond, and wherein cleavage of the selectively cleavable bond yields an Xpandomer of a length longer than the plurality of the XNTP subunits of the strand.

28. (canceled)

29. The method of claim 27, wherein the method further comprises: cleaving the selectively cleavable bonds to yield an Xpandomer.

30. The method of claim 27, wherein the reporter construct is used to parse genetic information in a sequence corresponding to the contiguous nucleotide sequence of all or a portion of the target nucleic acid, and the detecting changes in the voltage comprises detecting changes in the voltage due to the reporter constructs of the Xpandomer translocating the nanopore.

31. (canceled)

32. (canceled)

33. (canceled)
